(19) United States
(12) Patent Application Publication
(10) Pub. No.: US 2009/0025735 A1
(43) Pub. Date: Jan. 29, 2009

(54) MEDICAL GARMENT

(76) Inventor: Lynda Lavigne, Tarpon Springs, FL (US)

Correspondence Address: Maxey Law Offices, PLLC, 13630 58TH ST. NORTH, SUITE 101, CLEARWATER, FL, 33760, US

(21) Appl. No.: 11/782,849

(22) Filed: Jul. 25, 2007

(51) Int. Cl. A61F 5/37; A41D 1/04

(52) U.S. Cl. 128/876; 128/870; 2/102

(57) ABSTRACT

An apparatus for treating colic in infants including a garment having a garment body for wearing by the infant. A pair of bands extend latterly from opposite sides and in opposed directions from the garment for wrapping around the torso the infant. The bands have a width such that they extend from just below the underarms of the infant to the hips of the infant. The bands wrap around opposite sides of the infant's torso in front-to-back direction where the free ends of the bands are secured to the back of the garment. A pocket on the front of the garment is configured to receive a thermal pack to provide thermal therapy to the infant. In treating colic, the bands are wrapped snuggly about the infant's torso applying warmth and a slight pressure to thereby relieve discomfort.

### BACKGROUND OF THE INVENTION

1. Field of the Invention

The present invention relates generally to a medical garment for relieving pain and discomfort associated with various gastrointestinal ailments, and more particularly vest-like garment for relieving colic in infants.

2. Description of the Related Art

Colic is a fairly common condition in newborn infants; however, physicians and pediatricians alike are unsure of the exact cause of colic or where the pain stems from. Symptoms of colic typically include trapped stomach and intestinal gas, abdominal bloating, and acute gastric and intestinal pains. Colic is not only painful and uncomfortable for the infant it can be very stressful for the caregiver. It is not uncommon for a caregiver's health to decrease as a result of caring for a colicky baby. Further, the anxiousness of the caregiver can be sensed by the infant causing a worsening with the colic condition. Colic is typically treated by trying to determine the underlying cause and treating this cause. The problem with this approach is the exact cause of colic usually cannot determined, which results in a try-and-miss method of addressing various potential causes. This approach can be very frustrating for the caregiver lending to more stress.

Unfortunately for those infants that suffer from colic, its simply a part of the overall development and, in time, the infant will outgrow the condition. However, while the infant is suffering from colic, it is highly desirably to relieve the infant's pain and discomfort.

A device described in U.S. Pat. No. 5,518,009 provides a narrow belt for wrapping snuggly about an infant's lower torso, over the bellybutton region in an attempt to provide relief from the pain and discomfort. This device has drawbacks in that the belt is narrow and applies pressure only in a narrow localized area of the torso and the belt is prone to dislocation as a result of normal movement of the wearer. Additionally, the device of '009 requires the free ends of the belt to be overlap in a manner to engage the cooperating fasteners located on each end. During prolonged wear, the overlapping relationship of the ends of the belt can cause a sore spot to form on the wearer's torso requiring discontinued use of the belt until the sore heals.

As such there exists a desire and a need for an improved device for treating the symptoms of colic and providing relief to a suffer from the pain and discomfort caused by colic.

### SUMMARY OF THE INVENTION

The instant invention is constructed and designed to relive pain and discomfort associated with colic in a manner not previously known by providing a vest-like garment to be worn by the infant.

In general, in one aspect, a garment for treating colic infant is provided. The garment includes a garment body for wearing over the torso of a wearer, the garment body defining a front panel, a back panel, a head opening, a pair of arm openings, and a waist opening; a first band extending laterally from the front panel, the first band having a free end portion and dimensioned and configured for wrapping around one side of the torso of the wearer, the free end portion being mutually engagable with the back panel; a second band extending laterally from the front panel in an opposite direction of the first band, the second band having a free end portion and dimensioned and configured for wrapping around a second side of the torso of the wearer, the free end portion of the second band being mutually engagable with the back panel; and wherein the first band and the second band is of a width such that an upper edge of each band is located just underneath the underarm of the wearer and a lower edge of each band is located just above the hips of the wearer.

With the foregoing in mind, it is an object of the present invention to provide an apparatus for treating colic.

It is another object of the present invention to provide an apparatus for relieving colic in infants by applying a light pressure to the infants torso from just beneath the arms to just above the hips.

It is another object of the present invention to provide an apparatus for relieving colic in infants by applying a light pressure and heat to the infants torso from just beneath the arms to just above the hips.

It is another object of the present invention to provide an apparatus for relieving colic in infants which may be adjustably secured about the infant's torso in a manner to so as to apply a light pressure to the torso.

It is another object of the present invention to provide an apparatus including a garment to be worn by an infant for treating colic.

It is another object of the present invention to provide an apparatus for relieving colic in infants which does not create pressure sores.

It is another object of the present invention to provide an apparatus for relieving colic in infants which is easily put on the infant.

It is another object of the present invention to provide an apparatus for relieving colic in infants which will not dislocate during normal movement.

There has thus been outlined, rather broadly, the more important features of the invention in order that the detailed description thereof that follows may be better understood and in order that the present contribution to the art may be better appreciated.

Numerous other objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is an environmental, front elevation diagrammatic view of the present invention, showing the same in an unsecured configuration so as to more clearly illustrate the construction of the invention and how it is attached to an infant;

FIG. 2 is a rear elevation view of the present invention, also in an unsecured configuration;

FIG. 3 is a rear elevation view of the present invention in a secured configuration; and

FIG. 4 is a side elevation view of an alternate band construction.

Similar reference characters denote corresponding features consistently throughout the attached drawings.

### DETAILED DESCRIPTION OF THE INVENTION

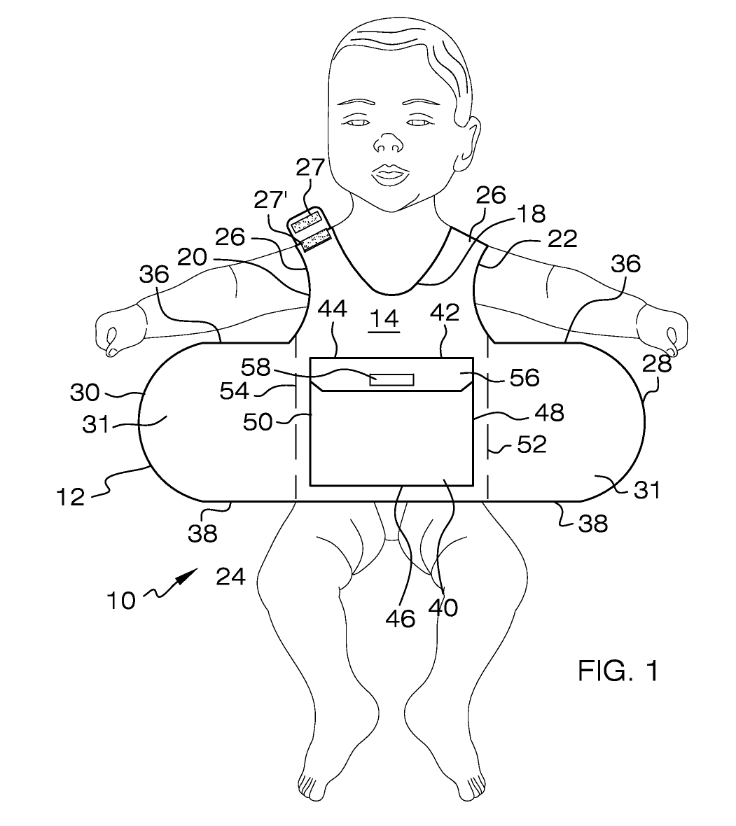

In FIG. 1 of the drawings, there is shown a preferred embodiment of a medical garment 10 that is dimensioned and configured to fit an infant. The garment 10 is shown worn by an infant in an unsecured configuration. The garment 10 has a garment body 12 that is generally vest-like and can be dimensioned to fit over an undergarment worn by the infant. The garment 10 is put on the infant as one would a pull-over shirt or the like.

It is important to note, while the description herein is made towards a garment to be worn by an infant for relieving the symptoms associated with colic, the applicant envisions the garment being used by children and adults alike to relieve pain and discomfort associated with many gastrointestinal ailments. As such, the garment can be configured and dimensioned to fit any person without departing from the true scope of the invention.

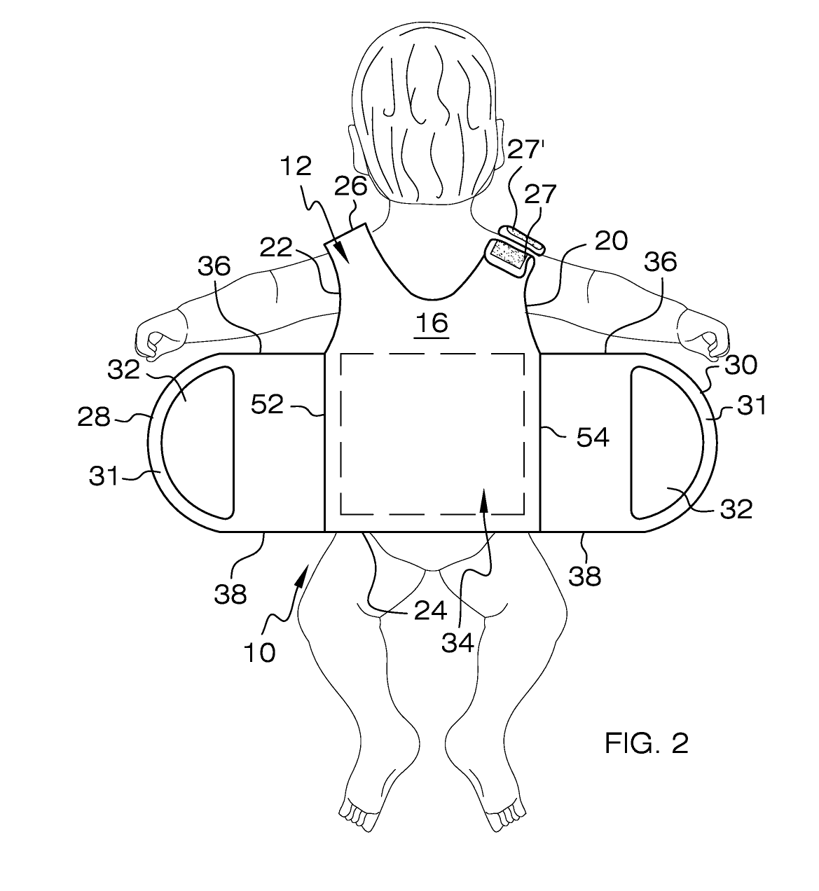

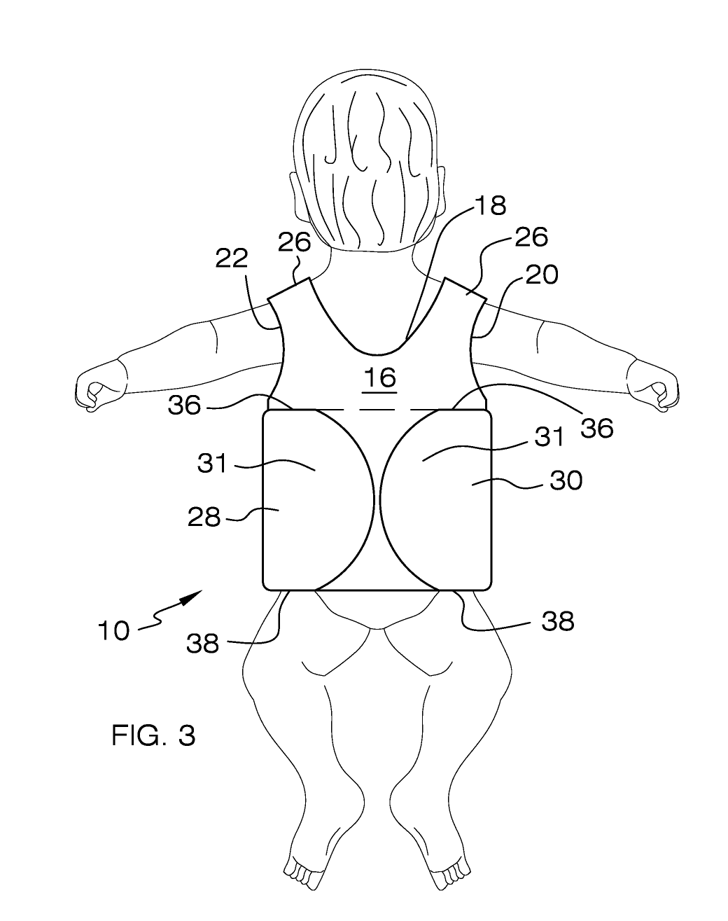

Referring now to FIGS. 1-3, the garment body 12 is a one-piece garment having a front panel 14 and a back panel 16, a head or neck opening 18, a pair of arm openings 20, 22 and a waist opening 24. The garment body 12 is constructed of a fabric that is preferably a hypo-allergenic fabric, such as cotton, which is both comfortable to the touch and breathable. The arm openings 20, 22 are defined by the garment body 12 to receive the arms of the wearer and form shoulder straps 26 which support the garment body over the torso of the wearer. One of the straps 26 can include two end portions 27 and 27′ that are mutually engagable with a mating fastener, such as for example, a hook-and-loop type fastener. The end portions 27, 27′ can be detached to facilitate quick remove of the garment in an emergency for example.

A pair of straps or bands 28, 30 extend laterally outward in opposed directions from the front panel 14. Each band 28, 30 has a free end portion 30, and is dimensioned and configured to wrap around the side of the wearer's torso from the side of the front panel 14 the band extends from in a direction from front to back. In other words, The first band 28 is dimensioned and configured to wrap around the left side of the wearer's torso, and the second band 30, is alike dimensioned and configured to wrap around the right side of the wearer's torso. The applicant has found the invention to be more effective when the bands 28 and 30 are configured to wrap around the wearer in a direction from the front to the back as opposed in a direction from the back to the front. It is believed a front-to-back wrapping direction not only applies a slight pressure to the wearer's torso but also acts to comfortably support and pull the abdomen inward increasing relief to the wearer.

The free end portions 31 are fitted with fasteners 32 that are engagable with the back panel 16 to secure the free end portions to the back panel. The fasteners 32 can be pile fasteners which are mutually engagable with the back panel in at least area 34. Alternatively, the free end portions 30 and the back panel 16 can be fitted with complementary fasteners, such as hook-and-loop fasteners. It is important to note, the bands 28 and 30 are dimensioned and configured so that the free end portions 30 do not overlap when secured to the back panel 16 while still applying a slight pressure to the wearer's torso, thereby preventing excess localized pressure that would otherwise result in a sore on the wearer's body.

The bands 28 and 30 are of width such that an upper edge 36 of each band is located just beneath the underarm of the wearer and a lower edge 38 of each band is located just above the wearer's hips. The bands 28 and 30 being of such a width insures a slight pressure is applied substantially across the wearer's torso from the upper chest to bellow the bellybutton, which is an important distinction from the prior art.

The bands 28 and 30 can be made integral with the front panel 14 and extend latterly outward from opposed vertical side edges of the front panel in opposite directions. Alternatively, a single band could be affixed to the front panel 14 leaving the two separate band portions 28 and 30 that extend laterally outward from the front panel in opposite directions. Alternatively yet, the each band 28 and 30 can be separate bands affixed to the front panel 14 so as to extend latterly outward from the front panel in opposite directions.

With particular reference to FIG. 1, a pocket 40 is affixed to the front panel 14. The pocket 40 is adapted to receive and hold a thermal pack (not shown) therein through an end opening 42. The thermal pack is held against the wearer's torso providing thermal therapy to increase relief from the colic. It is contemplated a reusable, and microwavable thermal pack containing a heat retaining material, such as flax-seed or the like, would be best suited for this application. Additionally, the thermal pack can include a soothing aroma therapy substance, such as lavender or the like.

Preferably, the pocket 42 is dimensioned and configured such that a top end 44 is located approximate the upper edge 36 of the bands 28, 30, a bottom end 46 is located approximate the lower edge 38, and opposed side ends 48, 50 are located approximate the vertical side edges 52, 54 of the front panel. The thermal pack would be similarly dimensioned to substantially span the area of the pocket 42. A closure 56, such as a flap having a complementary fastener 58 can be included to seal the end opening 42.

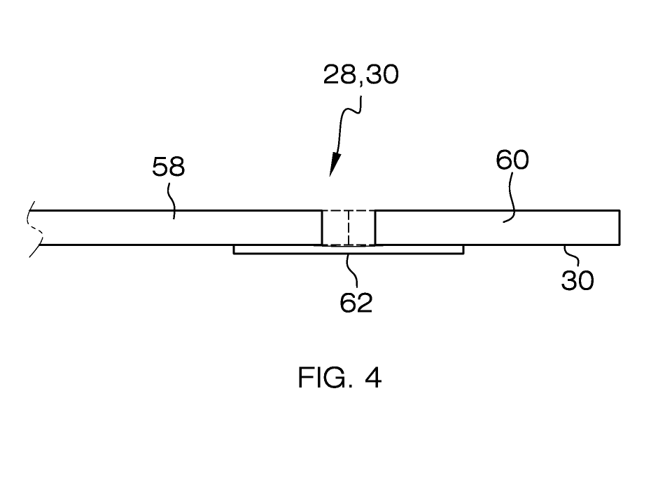

Now with reference to FIG. 4, an alternate construction of the bands 28 and 30 is shown. In this construction, and indication is provided to the caregiver that indicates when an appropriate amount of tension is applied to each band 28, 30 and thus an appropriate amount of pressure to the torso of the wearer. More particularly, each band 28 and 30 is comprised of to two separate band portions 58 and 60 that are interconnected at their ends by an intermediate elastic portion 62. The elastic portion 62 is configured such that when no tension is applied to the band 28, 30 the ends of the separate band portions 58 and 60 are abutted together. As tension is applied to the band 28, 30 by pulling on the end portion 30, the separate band portions 58 and 60 are pulled apart through stretching of the elastic portion 62. Once the appropriate amount of tension has been placed in the band 28, 30 a visual indication, such as a colored surface of the elastic portion is made visible. When to much tension has been placed in the band 28, 30 a second visual indication can be provided, such as a different colored surface, thereby indicating to much tension is in the band.

It is important to note, while the description herein has been made towards a garment to be worn by an infant for relieving the symptoms associated with colic, the applicant envisions the garment being used by children and adults alike to relieve pain and discomfort associated with many gastrointestinal ailments.

A number of embodiments of the present invention have been described. Nevertheless, it will be understood that various modifications may be made without departing from the spirit and scope of the invention. Accordingly, other embodiments are within the scope of the following claims.

I claim:

1. A garment for relieving gastrointestinal ailments, such as colic in infants, the garment comprising:
a garment body for wearing over the torso of a wearer, said garment body defining a front panel, a back panel, a head opening, a pair of arm openings, and a waist opening;
a first band extending laterally from said front panel; said first band having a free end portion and dimensioned and configured for wrapping around one side of the torso of the wearer, said free end portion being mutually engagable with said back panel;
a second band extending laterally from said front panel in an opposite direction of said first band, said second band having a free end portion and dimensioned and configured for wrapping around a second side of the torso of the wearer, said free end portion of said second band being mutually engagable with said back panel; and
wherein said first band and said second band is of a width such that an upper edge of each band is located just underneath the underarm of the wearer and a lower edge of each band is located just above the hips of the wearer.

2. The garment of claim 1, wherein said first band and said second bands are integral with said front panel.

3. The garment of claim 2, wherein said front panel has opposed first and second vertical edges and said first band extends from said first vertical edge and said second band extends from said second vertical edge.

4. The garment of claim 1, further comprising:
a pocket on said front panel, said pocket being dimensioned and configured to removably receive a thermal pack therewithin.

5. The garment of claim 4, wherein said pocket includes a closure for sealing said pocket.

6. The garment of claim 4, wherein said pocket has an upper end positioned approximate said upper edge of said first and said second bands, a lower end positioned approximate said lower edge of said first and said second bands, a first side end positioned approximate a first vertical side edge of said front panel, and a second side end positioned approximate a second vertical side edge of said front panel that is opposed said first vertical side edge.

7. The garment of claim 1, wherein said garment body is vest-like.
